(19) United States
(12) Patent Application Publication
(10) Pub. No.: US 2025/0134935 A1
(43) Pub. Date: May 1, 2025

(54) HERBAL SUPPLEMENTS AND METHODS OF USE AND MAKING THEREOF

(71) Applicant: KBS Research, LLC, Dallas, TX (US)

(72) Inventor: Kenneth Brown, Plano, TX (US)

(73) Assignee: KBS Research, LLC, Dallas, TX (US)

(21) Appl. No.: 18/494,134

(22) Filed: Oct. 25, 2023

(51) Int. Cl. A61K 35/742; A61K 35/00; A61K 36/22; A61K 36/534; A61K 36/77; A61P 1/10; A61P 1/14

(52) U.S. Cl. CPC A61K 35/742; A61K 36/22; A61K 36/534; A61K 36/77; A61P 1/10; A61P 1/14; A61K 2035/115

(57) ABSTRACT

An herbal supplement for reducing bloating, dysbiosis, diarrhea, constipation and/or weight gain in a human subject is disclosed. The herbal supplement comprises an herbal composition comprising condensed tannin; an herbal composition comprising a triterpenoid saponin or an anti-spasmodic agent; and one or more probiotics in endospore form, wherein the supplement is formulated to reduce bloating, dysbiosis, diarrhea, constipation and/or weight gain in a human subject.

### FIELD

The present application relates generally to herbal compositions and methods for treating diseases associated with bloating, constipation, diarrhea, microbiome imbalance and/or weight gain. More particularly, the present application relates to a dietary supplement for gastrointestinal health, specifically a combination of a polyphenol supplement and a spore-forming probiotic.

### BACKGROUND

Bowel disorders are often characterized by bloating, diarrhea or constipation and are thought to affect at least 20% of the population. Yet, to date no effective therapy is available. Such bowel disorders include irritable bowel syndrome (IBS), functional constipation, chronic pseudo-obstruction, and chronic abdominal bloating syndrome. Symptoms include abdominal pain, constipation, diarrhea, bloating, acid reflux, flatulence, nausea and vomiting, chronic lethargy and sleep disorders. Obesity is also an epidemic problem with few effective options.

Surveys have demonstrated that less than 14% of patients with IBS are satisfied with their treatment. Presently, there are few dependable pharmaceutical treatment options for IBS or obesity.

In view of the current shortcomings associated with bowel disorder treatments, including IBS and functional constipation, as well as obesity or undesired weight gain, there is a need for new treatments. The present application addresses this need and provides new methods and formulations for treating such disorders or diseases.

### SUMMARY

Gastrointestinal disorders, stemming from an imbalance in the microbiome (also referred to as “microbiome dysbiosis” or “dysbiosis”), have been found to correlate with various adverse health conditions, such as chronic systemic inflammation, increased intestinal permeability, neurodegenerative diseases, and cancer. The management of gastrointestinal and microbiome health thus represents a significant challenge in medical science. In response to this urgent need, the composition of the present application combines a polyphenol blend with a spore-biotic formulation. This combination harnesses the synergistic potential of these natural components, resulting in a multi-faceted solution for gastrointestinal health and microbiome restoration.

One aspect of the present application relates to an herbal supplement that comprises an herbal composition comprising condensed tannins, an herbal composition comprising a triterpenoid saponin or an anti-spasmodic agent; and one or more probiotics in endospore form, wherein the supplement is formulated to reduce bloating, dysbiosis, diarrhea, constipation and/or weight gain in a human subject.

Another aspect of the present application relates to a method for treating bloating, dysbiosis, diarrhea, constipation and/or weight gain in a human subject. The method comprises the step of orally administering to the human subject in need of such treatment an effective amount of the herbal supplement of the present application.

Another aspect of the present application relates to a method for preparing the herbal supplement of the present application. The method comprises the steps of mixing (1) red quebracho extract, (2) an herbal composition comprising a triterpenoid saponin and/or an anti-spasmodic agent; and (3) one or more probiotics in endospore form to product a mixture; and formulating the mixture into unit doses suitable for oral administration.

### DETAILED DESCRIPTION

The following detailed description is presented to enable any person skilled in the art to make and use the present application. For the purposes of explanation, specific nomenclature is set forth to provide a thorough understanding of the present application. However, it will be apparent to one skilled in the art that these specific details are not required to practice the present application. Descriptions of specific applications are provided only as representative examples. The present application is not intended to be limited to the embodiments shown, but is to be accorded the widest possible scope consistent with the principles and features discloses herein.

Unless otherwise defined, scientific and technical terms used in connection with the present application shall have the meaning that are commonly understood by those of ordinary skill in the art. Further, unless otherwise required by context, singular terms shall include pluralities and plural terms shall include the singular.

### I. Definitions

As used herein, the following terms shall have the following meanings: as used herein, the term “herbal composition” is used with reference to any phytochemical or mixture thereof that is obtained, isolated, and/or derived from one or more extracts of plant material(s) or essential oil(s) thereof. The term “Plant material” refers to any plant material including, but not limited to, leaves, hark, stems, flowers, fruits, seeds, roots, and combinations thereof. The terms “herbal extract,” and “plant extract” are used interchangeably with reference to a plant material directly extracted from a plant. An extract may be in the form of a dry powder, solution or oil.

As used herein, the phrase “treating” or “treatment” of weight gain is synonymous with promotion of weight loss, as well as controlling or managing body weight, or more specifically, prevention of weight gain and/or inhibiting the promotion of weight gain.

The term “nutraceutically acceptable,” such as in the recitation of a “nutraceutically acceptable carrier,” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained.

An “effective amount” refers to a nontoxic but sufficient amount of a composition or plant material to provide a desired systemic or local effect. The effective amount will vary with the nature of the composition and constituent parts, the age and physical condition of the end user, the severity of the bowel disorder, the duration of the treatment, the nature of concurrent therapy, the particular pharmaceutically acceptable carrier utilized, and like factors. As used herein, all percentages are by weight unless otherwise specified.

As used herein, the transitional term “comprising” and “comprises” are synonymous with “including”, “containing” or “characterized by”, any one of which is inclusive or open-ended and does not exclude additional, unrecited elements or method steps, regardless of its use in the preamble or the body of a claim. The term further encompasses the terms “consisting of” and “consisting essentially of”. In the claims and/or specification, “comprising” may mean “one”, but it is also consistent with the meaning of “one or more”, “at least one” and “one or more than one”.

As used herein, the phrase “a composition that is substantially free of X” means that component X is present in the composition in a trace amount of less than 1% (w/w), 0.1% (w/w) or 0.01% (w/w).

The term “plant material” refers to any plant material including, but not limited to, leaves, hark, stems, flowers, fruits, seeds, roots, and combinations thereof.

The terms “herbal extract,” and “plant extract” are used interchangeably with reference to a composition extracted from a plant. An herbal extract may be in the form of a dry powder, solution or oil (such as essential oils). An herbal extract may contain a mixture of components from the original plant material. An herbal extract may also be in a partially purified form enriched for certain component or components in the original plant.

As used herein, the term “extract ratio” refers to a weight ratio between the raw material and the final extract. The higher is the extract ratio, the more concentrated is the extract. For example, an extract with a 10:1 extract ratio means that 10 parts of the raw material were used to produce 1 part of the extract.

### II. Composition

One aspect of the present application relates to an herbal supplement for treating bowel disorders associated with bloating, constipation, diarrhea, dysbiosis and/or weight gain. In some embodiments, the herbal supplement comprises (1) an herbal composition containing condensed tannin, (2) an herbal composition containing a triterpenoid saponin and/or an anti-spasmodic agent, and (3) one or more probiotics in endospore form, wherein the herbal supplement is formulated to reduce bloating, constipation, diarrhea, dysbiosis and/or weight gain in a human subject.

In some embodiments, the herbal supplement comprises (1) an herbal composition containing condensed tannin, (2) an herbal composition containing a triterpenoid saponin, (3) an anti-spasmodic agent, and (4) one or more probiotics in endospore form.

The herbal supplement of the present application may be used to treat or alleviating symptoms of any bowel disorder characterized by bloating, constipation, diarrhea, and/or dysbiosis, as well as obesity and other diseases characterized by unwanted weight gain.

While not to be limited by the theory, it is believed that the herbal supplement of the present application may achieve one or more of the following effects:

### (1) Decrease Intestinal Permeability.

The bacteria strains in the herbal supplement of the present application decrease intestinal permeability by one or more of the following mechanisms:
(a) suppressing growth of pathogenic bacteria such as E. coli and Salmonella that can damage tight junction proteins and cause intestinal inflammation;
(b) increasing production of short-chain fatty acids like butyrate, which provide energy to intestinal cells and strengthen tight junctions;
(c) stimulating mucin synthesis, thickening the mucus layer protecting epithelial cells. Mucins also contain proteins that support tight junctions;
(d) modulating expression of key tight junction proteins like occludin, claudin, and ZO-1, closing gaps between enterocytes;
(e) reducing intestinal permeability triggered by toxins, pathogens, or pro-inflammatory cytokines by stabilizing actin and myosin rings around enterocytes; and
(f) inhibiting apoptosis and preserves mucosal integrity by downregulating programmed cell death pathway.

### (2) Modulate LPS Signaling.

The bacteria strains in the herbal supplement of the present application exhibit a remarkable ability for LPS degradation, and may play a crucial role in neutralizing bacterial endotoxins and subsequently downregulating inflammatory cascades.

### (3) Regulate Cytokine Production

The bacteria strains in the herbal supplement of the present application may act as regulators of cytokine expression, exhibiting an inhibitory effect on pro-inflammatory cytokines and concurrently promoting anti-inflammatory cytokines.

### (4) Produce Microbiome Effect

Through the synergism of the polyphenols and the bacteria strains in the herbal supplement of the present application, the large polyphenols are broken down into smaller phenolic compounds which are then able to be used as prebiotics and improve microbial diversity. Both the polyphenols and spore biotics decrease harmful bacteria and increase helpful bacteria, such as Akkermansia muciniphila.

### (5) Increase Antioxidants

The bacteria strains in the herbal supplement of the present application may create optimal conditions for the bioactivity of phenolic compounds which results in increased antioxidant activity.

### (6) Disrupt Bacterial Biofilms

The tannins in the polyphenols in the herbal supplement of the present application have numerous mechanisms by which they can inhibit the formation and stability of bacterial biofilms. The broad protein binding activity of tannins allows them to target many critical components in the biofilm matrix and bacterial surfaces, thus disrupting the formation of bacterial biofilms.

### Herbal Composition Comprising Condensed Tannins

Condensed tannins (proanthocyanidins, polyflavonoid tannins, catechol-type tannins, pyrocatecollic type tannins, non-hydrolyzable tannins or flavolans) are a complex group of polyphenolic compounds found in a wide variety of plant species. Condensed tannins usually have a molecular weight of about 1-20 kDa.

In some embodiments, the herbal composition comprising triterpenoid saponin is provided in the form of a plant extract. Plant parts containing tannins include bark, wood, fruit, fruit pods, leaves, roots and plant galls. Condensed tannins may be provided in the form of plant extracts derived from selected plants (and their representative members), including but not limited to birch (Belula sp.), canaigre (Rumex hymenocephalus), chestnut wood (Casanea sp., incl. saliva and dentata), Eastern hemlock (Tsuga canadensis), eucalyptus (Eucalyptus sp.), European larch (Latrix decidua), mangrove (Rhizophora sp., incl. mangle), oak (Quercus sp., incl. montana), pine (Pinus sp.), pomegranate (Punica granatum), quebracho woods (Schniposis sp.), rhatany root (Krameria triandra), Scotch pine bark (Pinus sylvestris), spruce (Picea sp., incl. abies), sumac (Rhus sp.), wattle (Acacia sp., incl. decurrens and mearnsii), willow (Salix caprea), willow bark, wine grape seed (Vitis vinifera), cinnamon bark, Eucommia ulmoides (Eucommia bark), Swedish birch bark, canaigre root, Emblica officinalis (Amla/Indian gooseberry), Belliric myrabolam/Terminalia bellirica (Bibhitaki fruit), Acacia mearnsii/Acacia senegal/Acacia seyal (black wattle), Terminalia chebula (Haritaki fruit), Mimosa extract (Mimosa pigra/Mimosa pudica), acorn, chestnut, paulownia, persimmon, birch leaf, and walnut leaf.

In some embodiments of the present application, the herbal composition containing condensed tannins comprises a red quebracho extract. The red quebracho extract may be derived from any red quebracho tree, including but not limited to Shinopsis lorentzii, Schinopsis balansae, Schinopsis brasiliensis, Schinopsis haenkeana, Schinopsis heterophylla and Schinopsis marginaia. In one embodiment, the red quebracho extract is prepared from the bark of a red quebracho tree.

In some embodiments, the red quebracho extract comprises at least 25%, at least 40%, at least 50%, at least 60%, at least 70%, at least 80% or at least 90% or more condensed tannins (w/w). In some embodiments, the red quebracho extract is substantially free of hydrolyzable tannins. In some embodiments, the red quebracho extract is in the form of a powder. In some embodiments, the red quebracho extract is in the form of a powder with a condensed tannin content of at least 80%. In some embodiments, the red quebracho extract is in the form of a powder, substantially free of hydrolysable tannins and having a condensed tannin content of at 50%, at least 60%, at least 70% or about 73%.

The red quebracho extract may be present in any amount sufficient for treating bloating or constipation. In some embodiments, the herbal supplement of the present application is in a dosage form that comprises red quebracho extract in an amount between about 10 to about 1000 mg, between about 25 to about 500 mg, between about 50 to 250 mg, or between about 100 to 200 mg. In some embodiments, the herbal supplement of the present application is in a dosage form that contains 160 mg red quebracho extract.

In some embodiments, the quebracho extract has an extract ratio of 1:1, 2:1, 3:1, 4:1, 5:1, 6:1, 7:1, 8:1, 9:1, 10:1, 15:1 or 20:1.

### Herbal Composition Comprising a Triterpenoid Saponin

Triterpenoid saponins belong to a large group of structurally diverse surface active glycoside compounds that found in a wide variety of plant species. Triterpenoid saponin compounds typically contain sugars moieties in a four or five ring configuration of about 30 carbons with several oxygens attached.

In some embodiments, the herbal compositions comprising triterpenoid saponin is provided in the form of a plant extract, such as an Aesculus or Sapindus species plant extract. In some embodiments, the herbal compositions comprising triterpenoid saponin is provided in the form of an extract of horse chestnut (Aesculus hippocas[anum) or an extract of soapnut (Satindus trifoliatus), including seed extracts thereof.

The Triterpenoid saponins may be present in any amount sufficient for treating bloating or constipation. In some embodiments, the herbal supplement of the present application is in a dosage form that comprises an extract of horse chestnut or an extract of soapnut, in an amount between about 5 to about 500 mg, between about 10 to about 250 mg, or between about 25 to about 100 mg. In some embodiments, the herbal supplement of the present application is in a dosage form that contains 45 mg chestnut extract.

In some embodiments, the horse chestnut extract has an extract ratio of 1:1, 2:1, 3:1, 4:1, 5:1, 6:1, 7:1, 8:1, 9:1, 10:1, 15:1 or 20:1.

### Anti-Spasmodic Agents

Anti-spasmodic (or spasmolytic) agents prevent or ease spasms or cramps in muscles, and provide particular benefit in the muscles of the gut and bladder of IBS patients, especially with regard to their ability to reduce abdominal pain, calm and sooth the digestive system, relax gastrointestinal smooth muscles, relax the gastro-esophageal sphincter and to increase time and absorption of therapeutic agents into the small intestine. A wide variety of plants are known to naturally synthesize anti-spasmodic agents.

In some embodiments, the anti-spasmodic agent is provided in the form of an herbal extract derived from selected plants (and their representative members), including but not limited to barberry (Berberis vulgaris), basil (Ocimum basilicum), black cohosh (Actaea racemosa), centella (Centella asiatica), chamomile (Matricaria recutita), cramp bark (Viburnum opulus), dill (Anethum graveolens), fennel (Foeniculum vulgare), ginger (Zingiber officinale), hawthorn (Crataegus monogyna), hops (Humulus lupulus), juniper berries (Juniperus communis), lemon balm (Melissa officinalis), licorice (Glycyrrhiza glabra), marshmallow (Althaea officinalis), nutmeg (Myristicafragrans), peppermint (Mentha piperita), rosemary (Rosmarinus offinalis), saffron (Crocus salivus), sage (Salvia officinalis), skullcap, (Scutellaria baicalensis), slippery elm (Ulmus' rubra), spearmint (Menlha spicata), thyme (Thymus vulgaris), valerian (Valeriana officinale), wild lettuce (Lac[uca virosa) and wild yam (Dioscorea villosa).

In some embodiment, the anti-spasmodic agent is provided in the form of peppermint extract, a well know flavoring agent derived from the leaves and flowering tops of the Mentha piperita L plant, a hybrid mint that is a cross between water mint and spearmint. In some embodiments, the herbal supplement of the present application is in a dosage form that comprises peppermint oil, in an amount between about 1 to about 100 mg, between about 2 to about 50 mg, or between about 5 to about 200 mg. In some embodiments, the herbal supplement of the present application is in a dosage form that contains about 10 mg peppermint oil.

### Probiotics in Endospore Form

Probiotics in endospore form, also known as sporebiotics and spore-forming probiotics, belong to a type of dietary supplement that contains beneficial bacteria in the form of endospores. They are a specific group of microorganisms that can form a unique protective spore shell when conditions are unfavorable. This allows them survival in harsh conditions, such as the acidic environment of the stomach, enabling the spores to reach the intestines. Once in the intestines/colon they can germinate and transition into their active vegetative state, colonize the gut lining, and interact with the surrounding environment to exert their beneficial effects.

In some embodiments, the one or more probiotics in the herbal supplement of the present application are selected from the group consisting of Bacillus subtilis, Bacillus coagulans, Bacillus clausii, Bacillus cereus, Bacillus licheniformis, Bacillus polyfermenticus, Bacillus pumilus, Bacillus amyloliquefaciens, Bacillus siamensis, Bacillus tequilensis, Bacillus megaterium, and Bacillus aryabhattai

In some embodiments, the one or more probiotics are present in the herbal supplement of the present application in an individual or total amount in the range of 3.5×10⁸ to 3.5×10¹⁰ colony forming unit (cfu) or 1×10⁹ to 1×10¹⁰ cfu. In some embodiments, the one or more probiotics are present in the herbal supplement of the present application in an individual or total amount of about 3.5×10⁹ cfu.

5×10⁹ to 20×10⁹ colony forming units (CFU) per dose, more preferably between 1×10⁹ to 15×10⁹ CFUs per dose, more preferably between 3×10⁹ to 9×10⁹ CFUs per dose.

In some embodiments, the herbal supplement of the present application comprises Bacillus coagulans, in endospore form. In some embodiments, the herbal supplement of the present application comprises Bacillus clausii in endospore form.

In some embodiments, the herbal supplement of the present application comprises two or more probiotics in endospore form, wherein the two or more probiotics are selected from the group consisting of of Bacillus subtilis, Bacillus coagulans, Bacillus clausii, Bacillus cereus, Bacillus licheniformis, Bacillus polyfermenticus, Bacillus pumilus, Bacillus amyloliquefaciens, Bacillus siamensis, Bacillus tequilensis, Bacillus megaterium, and Bacillus aryabhattai.

In some embodiments, the herbal supplement of the present application comprises Bacillus subtilis and Bacillus coagulans in endospore form. In some embodiments, the herbal supplement of the present application comprises Bacillus subtilis and Bacillus clausii in endospore form. In some embodiments, the herbal supplement of the present application comprises Bacillus coagulans and Bacillus clausii in endospore form. In some embodiments, the herbal supplement of the present application comprises two probiotics in endospore form, wherein the two probiotics are present in the herbal supplement at a cfu/cfu or w/w ratio of 10:1 to 1:10.

In some embodiments, the herbal supplement of the present application comprises three or more probiotics in endospore form, wherein the two or more probiotics are selected from the group consisting of of Bacillus subtilis, Bacillus coagulans, Bacillus clausii, Bacillus cereus, Bacillus licheniformis, Bacillus polyfermenticus, Bacillus pumilus, Bacillus amyloliquefaciens, Bacillus siamensis, Bacillus tequilensis, Bacillus megaterium, and Bacillus aryabhattai.

In some embodiments, the herbal supplement of the present application comprises Bacillus subtilis, Bacillus coagulans and Bacillus clausii in endospore form. In some embodiments, the herbal supplement of the present application comprises three probiotics in endospore form, wherein the the probiotics are present in the herbal supplement at a cfu/cfu or w/w ratio of about 1:1:1. In some embodiments, the herbal supplement of the present application comprises Bacillus subtilis, Bacillus coagulans and Bacillus clausii in endospore form in a total amount of in the range of 1×10⁹ to 1×10¹⁰ cfu. In some embodiments, the herbal supplement of the present application comprises Bacillus subtilis, Bacillus coagulans and Bacillus clausii in endospore form in a total amount of about 3.5×10⁹ cfu.

### Component Weight Ratios

In some embodiments, the herbal supplement of the present application comprises quebracho extract, horse chestnut extract, and one or more probiotics in endospore form, wherein the quebracho extract and horse chestnut extract are present in the herbal supplement at a weight ratio in the range of 1:1 to 10:1, 2:1 to 6:1 or 3:1 to 4:1.

In some embodiments, the herbal supplement of the present application comprises quebracho extract, peppermint extract, and one or more probiotics in endospore form, wherein the quebracho extract and peppermint extract are present in the herbal supplement at a weight ratio in the range of 200:1 to 1:1, 160:1 to 2:1, 100:1 to 4:1, 50:1 to 6:1 25:1 to 8:1 or 20:1 to 10:1.

In some embodiments, the herbal supplement of the present application comprises quebracho extract, chestnut extract, peppermint extract, and one or more probiotics in endospore form, wherein the chestnut extract and peppermint extract are present in the herbal supplement at a weight ratio in the range of 50:1 to 1:2, 20:1 to 1:1, or 10:1 to 2:1.

In some embodiments, the herbal supplement of the present application comprises quebracho extract, chestnut extract, peppermint extract, and one or more probiotics in endospore form, wherein the herbal supplement is formulated in a dosage form containing 30-800 mg quebracho extract, 10-200 mg horse chestnut extract, 2-50 mg pepper mint extract and a mixture of Bacillus subtilis, Bacillus coagulans and Bacillus clausii in endospore form in a total amount of in the range of 1×10⁹ to 1×10¹⁰ cfu.

In some embodiments, the herbal supplement of the present application comprises quebracho extract, chestnut extract, peppermint extract, and one or more probiotics in endospore form, wherein the herbal supplement is formulated in a dosage form containing 80-320 mg quebracho extract, 20-90 mg horse chestnut extract, 5-20 mg pepper mint extract and a mixture of Bacillus subtilis, Bacillus coagulans and Bacillus clausii in endospore form in a total amount of in the range of 2×10⁹ to 6×10⁹ cfu.

In some embodiments, the herbal supplement of the present application comprises quebracho extract, chestnut extract, peppermint extract, and one or more probiotics in endospore form, wherein the herbal supplement is formulated in a dosage form containing 160 mg quebracho extract, 45 mg horse chestnut extract, 10 mg pepper mint extract and a mixture of Bacillus subtilis, Bacillus coagulans and Bacillus clausii in endospore form in a total amount of in the range of 3.5×10⁹ cfu.

### Additional Components

In some embodiments, the herbal supplement of the present application further comprises a nutraceutically acceptable carrier, such as cellulose.

### III. Method of Treatment

Another aspect of the present application relates to a method for treating or alleviating symptoms of any bowel disorder characterized by bloating, diarrhea, constipation and/or dysbiosis. Exemplary bowel disorders for treatment include irritable bowel syndrome (IBS), functional constipation, and chronic abdominal bloating syndrome.

In certain embodiments, the compositions and methods relate to treating one or more symptoms of an IBS disease subtype, including bloating and/or constipation. According to the Rome Ill classification system, there are four recognized subtypes of IBS: a) constipation predominant IBS (IBS-C), characterized by hard or lumpy stools in 225% of bowel movements and loose (mushy) or watery stools in <25% of bowel movements; b) diarrhea-predominant IBS (IBS-I)), characterized by loose (mushy) or water stools in 225% of bowel movements and hard or lumpy stools in <25% of bowel movements; and c) mixed-type IBS (IBS-M; sometimes diarrhea, sometimes constipation), characterized by hard or lumpy stools in 225% of bowel movements and loose (mushy) or watery stools in 225% of bowel movements; and d) unsubtyped IBS (IBS-U), characterized by insufficient abnormality of stool consistency to meet the criteria for IBS-C, D, or M. Alternating IBS (IBS-A) accounts for a large proportion of patients whose bowel habit oscillate from diarrhea to constipation and vice versa, such that up to 75% of patients (or more) transition between IBS-D and IBS-C over a I year period.

Additional symptoms that may be treated or alleviated by the herbal supplement of the present application include abdominal pain, abdominal fullness, abdominal distension; abnormal stool frequency, i.e., fewer than three bowel movements per week or more than three bowel movements per day; hard or lumpy stools, sometimes loose (mushy) or watery stools; straining during a bowel movement; urgency (having to rush to have a bowel movement); feeling of incomplete bowel movement; or passing of mucus during a bowel movement.

In a further aspect, the present application provides a method for managing or controlling weight gain or obesity in a human subject. The method comprises orally administering to the human subject in need thereof an effective amount of the herbal of the present application.

In a further aspect, the present application provides a method for managing or alleviating one or more symptoms of type Il diabetes in a human subject. The method comprises orally administering to the human subject in need thereof an effective amount of the herbal of the present application.

In some embodiments, the herbal composition is administered orally. The unit dose and frequency of administration may vary. In some embodiments, the herbal composition is administered twice a day, once a day, once every two days, once every three days, once every four days or once a week. In some embodiments, the herbal composition is administered for a period of one week, two weeks, three weeks, one month, two months of three months or for period time until alleviation of one or more symptoms of the target disease, disorder or condition.

### IV. Method of Making

The herbal supplement may be prepared using any suitable plant extract(s) and spore biotics.

Extracts for use in the present application may be produced from plant tissues/parts that can be extracted by water, polar, or petroleum solvents for treating irritable bowel syndrome. An extract may be prepared using extraction procedures well known in the art (e.g., the use of organic solvents such as lower alcohols, alkyl esters, alkyl ethers, alkyl ketones, chloroform, petroleum ether, hexane and/or inorganic solvents such as water). Additionally, the extracts may be produced by cold extraction techniques using a variety of different extraction solvents including, but not limited to, water, fatty solvents (such as olive oil), and alcoholic solvents (e.g. 70% ethanol). Cold extraction techniques may be applied to softer parts of the plant such as leaves and flowers, or in cases wherein the desired active components of the plant are heat labile. Alternatively, the aforementioned solvents may be used to produce extracts of the desired plants by a hot extraction technique, wherein said solvents are heated to a high temperature, the precise value of said temperature being dependent on the properties of the chosen solvent, and maintained at that temperature throughout the extraction process. Hot extraction techniques are more commonly applied to the harder, tougher parts of the plant, such as bark, woody branches and larger roots. In some cases, sequential extractions may be performed in more than one solvent, and at different temperatures.

Standard procedures for producing plant extracts (including hot extraction, cold extraction and other techniques) are described in many publications including “Medicinal plants: a field guide to the medicinal plants of the Land of Israel (in Hebrew), author: N. Krispil, Har Gilo, Israel, 1986” and “Making plant medicine, author: R. Cech, pub. by Horizon Herbs, 2000,” which are incorporated herein by reference in their entirety. Preferably, the supplement comprises one or more plant extract(s) prepared in powdered form.

In certain embodiments, the supplement may include an herbal extract in the form of an essential oil. As used herein, the term “essential oil” refers to an oil derived from herbs or other plants through steam distillation or cold pressing. An essential oil may be mixed with a vegetable oil or water and may be orally ingested for use in accordance with the present disclosures.

In some cases, leaves from a given plant species may be steam distilled to make the essential oil from that plant species. The essential oil made from a defined tree species may be used interchangeably for the name of the plant species and for the name of the essential oil produced from the leaves and other components of the plant species. Likewise, the same applies to an extract derived from a given plant species.

The one or more probiotics in endospore form may be obtained by subjecting the one or more probiotics to unfavorable environmental conditions. Endospore formation begins when the bacteria experience stressful conditions like lack of nutrients. This triggers them to undergo sporulation, which is the process of transforming into spore form. The spore becomes dormant, dehydrated and metabolically inactive. When conditions become favorable again, germination can occur rapidly, returning the endospore to its vegetative bacterial state.

The herbal supplement can be provided in any nutraceutically acceptable form. Preferably, the herbal supplement is formulated for oral administration as, for example but not limited to, drug powders, crystals, granules, small particles (which include particles sized on the order of micrometers, such as microspheres and microcapsules), particles (which include particles sized on the order of millimeters), beads, microbeads, pellets, pills, microtablets, compressed tablets or tablet triturates, molded tablets or tablet triturates, and in capsules, which are either hard or soft and contain the composition as a powder, particle, bead, solution or suspension.

The herbal supplement can also be formulated for oral administration as a solution or suspension in an aqueous liquid, as a liquid incorporated into a gel capsule or as any other convenient formulation for administration, or for rectal administration, as a suppository, enema or other convenient form. In addition, the compositions of the application can also be provided as a controlled release system.

The herbal supplement may further include any nutraceutically acceptable excipient, carrier or mixture thereof. As used herein, the term “nutraceutically acceptable excipient or carrier” refers to a non-toxic, inert solid, semi-solid, diluent, encapsulating material or formulation auxiliary of any type. Exemplary excipients include, but are not limited to diluents or fillers, such as dextrates, dicalcium phosphate, calcium sulfate, lactose, cellulose, kaolin, mannitol, sodium chloride, dry starch, sorbitol, sucrose, inositol, powdered sugar, bentonite, microcrystalline cellulose, or hydroxypropylmethylcellulose may be added to the inhibitor molecule to increase the bulk of the composition. Also, binders, such as but not limited to, starch, gelatin, sucrose, glucose, dextrose, molasses, lactose, acacia gum, sodium alginate, extract of Irish moss, panwar gum, ghatti gum, mucilage of isapgol husks, carboxymethylcellulose, methylcellulose, polyvinylpyrrolidone, Veegum and starch arabogalactan, polyethylene glycol, ethylcellulose, and waxes, may be added to the supplement to increase its cohesive qualities. Additionally, lubricants, such as but not limited to, talc, magnesium stearate, calcium stearate, stearic acid, hydrogenated vegetable oils, polyethylene glycol, sodium benzoate, sodium acetate, sodium chloride, leucine, carbowax, sodium lauryl sulfate, and magnesium lauryl sulfate may be added to the supplement. Also, glidants, such as but not limited to, colloidal silicon dioxide or talc may be added to improve the flow characteristics of a powdered supplement. Finally, disintegrants, such as but not limited to, starches, clays, celluloses, algins, gums, crosslinked polymers (e.g., croscarmelose, crospovidone, and sodium starch glycolate), Veegum, methylcellulose, agar, bentonite, cellulose and wood products, natural sponge, cation-exchange resins, alginic acid, guar gum, citrus pulp, carboxymethylcellulose, or sodium lauryl sulfate with starch may also be added to facilitate disintegration of the supplement in the intestine.

In certain embodiments, one or more compositions of the supplement may be formulated to protect the composition from degradation by the acidic conditions of the stomach and from interactions with proteins, such as pepsin, present in the stomach. Such a formulation may include a pH-dependent enteric coating to prevent release until after gastric emptying. Thus, in some embodiments, one or more compositions or the entire supplement is enteric coated. However, in other embodiments no compositions of the supplement are enteric coated.

An enteric coated composition or supplement may be formulated as enteric coated tablets, beads or granules, which may optionally contain a lubricant such as, but not limited to, magnesium stearate. The enteric coating may include one or more pH dependent polymers. The pH dependent polymers may remain intact at pH values lower than about 4.0 and dissolve at pH values higher than 4.0, preferably higher than 5.0, most preferably about 6.0. Exemplary pH-dependent polymers include, but are not limited to, methacarylic acid copolymers, methacrylic acid-methyl methacrylate copolymers (e.g., EUDRAGIT® L100 (Type A), EUDRAGIT® S100 (Type B), Rohm GmbH, Germany; methacrylic acid-ethyl acrylate copolymers (e.g., EUDRAGIT® L100-55 (Type C) and EUDRAGIT® L30D-55 copolymer dispersion, Rohm GmbH, Germany); copolymers of methacrylic acid-methyl methacrylate and methyl methacrylate (EUDRAGIT® FS); terpolymers of methacrylic acid, methacrylate, and ethyl acrylate; cellulose acetate phthalates (CAP); hydroxypropyl methylcellulose phthalate (HPMCP) (e.g., HP-55, HP-50, HP-55S, Shinetsu Chemical, Japan); polyvinyl acetate phthalates (PVAP) (e.g., COATERIC®, OPADRY® enteric white OY-P-7171); polyvinylbutyrate acetate; cellulose acetate succinates (CAS); hydroxypropyl methylcellulose acetate succinate (HPMCAS), e.g., HPMCAS LF Grade, MF Grade, HF Grade, including AQOAT® LF and AQOAT® MF (Shin-Etsu Chemical, Japan); Shinetsu Chemical, Japan); shellac (e.g., MARCOAT™ 125 & MARCOAT™ 125N); vinyl acetate-maleic anhydride copolymer; styrene-maleic monoester copolymer; carboxymethyl ethylcellulose (CMEC, Freund Corporation, Japan); cellulose acetate phthalates (CAP) (e.g., AQUATERIC®); cellulose acetate trimellitates (CAT); and mixtures of two or more thereof at weight ratios between about 2:1 to about 5:1, such as, for instance, a mixture of EUDRAGIT® L 100-55 and EUDRAGIT® S 100 at a weight ratio of about 3:1 to about 2:1, or a mixture of EUDRAGIT® L 30 D-55 and EUDRAGIT® FS at a weight ratio of about 3:1 to about 5:1.

The pH dependent polymers can be incorporated in an amount from about 10% to 90%, preferably from about 20% to 80% and most preferably from about 30% to 70% by weight of the dosage unit or supplement. The polymer(s) can be incorporated into the formulation either prior to or after granulation or they can be added into the supplement either as a dry material, or they can be dispersed or dissolved in an appropriate solvent, and dispersed during granulation.

An enteric coated composition or supplement may include enteric coated beads in a capsule, enteric coated microspheres in a capsule, enteric coated microspheres provided in a suspension or mixed with food, which are particularly convenient for pediatric administration, and enteric coated compressed tablets. The capsule can be a hard-shell gelatin capsule or a cellulose capsule. In particular, the composition or herbal supplement may be formulated as an enteric coated capsule.

In some embodiments, the composition(s) and/or supplements may be directly compressed, with or without any excipients, into a tablet or other herbal supplement having a nutraceutically acceptable hardness and friability. Preferably, the directly compressible herbal supplement can be compressed into tablets having a hardness of greater than 4 kp (kiloponds), preferably a hardness of 8 to 14 kp, more preferably a hardness of 10 to 13 kp. A directly compressible composition can be compressed into a tablet that has a friability of not more than 1% loss in weight, preferably less than 0.8% loss in weight, more preferably less than 0.5% loss in weight.

The herbal supplement may comprises one or more extracts separately administered in combination with one another (e.g. in the form of capsules or tablets) or they may be administered together in a single formulation. In some embodiments, the herbal supplement is administered in a capsule form, wherein each capsule comprises a mixture of a red quebracho extract, a horse chestnut extract, a peppermint extract, and a miturx of Bacillus subtilis, Bacillus coagulans and Bacillus clausii in endospore form.

Compositions and medicaments containing mixtures of extracts from different plant species, such as those of the present application may be prepared using different ratios of each extract.

The herbal supplement can be provided in any nutraceutically acceptable form. Preferably, the herbal supplement is formulated for oral administration as, for example but not limited to, drug powders, crystals, granules, small particles (which include particles sized on the order of micrometers, such as microspheres and microcapsules), particles (which include particles sized on the order of millimeters), beads, microbeads, pellets, pills, micro-tablets, compressed tablets or tablet triturates, molded tablets or tablet triturates, and in capsules, which are either hard or soft and contain the composition as a powder, particle, bead, solution or suspension.

The present application is further illustrated by the following Example that should not be construed as limiting.

### EXAMPLES

### Example 1: The Herbal Supplement of the Present Application Is Effective in Alleviating Bloating and Constipation

A trial batch of the herbal supplement (test supplement) of the present application was prepared by mixing quebracho extract, horse chestnut extract, peppermint oil, and a probiotics mixture to produce a final mixture with at quebracho extract: horse chestnut extract: peppermint oil weight ratio of 32:9:2 and about 3.5×10⁹ cfu probiotics in endospore form per unit dose.

A trail was conduct with about 150 patients who had experienced symptoms of bloating, diarrhea or constipation, and who had been treated with an herbal supplement (control supplement) that contains quebracho extract, horse chestnut extract and peppermint oil, but no probiotics. The trial patients switched from the control supplement to the test supplement and reported alleviation of symptoms of bloating and constipation. About 60% of the patients under trial decided to stay with the test supplement, despite of the increase cost.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. An herbal supplement, comprising:
an herbal composition comprising condensed tannins;
an herbal composition comprising a triterpenoid saponin or an anti-spasmodic agent; and
one or more probiotics in endospore form,
wherein the supplement is formulated to reduce bloating, dysbiosis, diarrhea, constipation and/or weight gain in a human subject.

2. The herbal supplement of claim 1, wherein the one or more probiotics comprise one or more species of Bacillus.

3. The herbal supplement of claim 2, wherein the one or more species of Bacillus are selected from the group consisting of Bacillus subtilis, Bacillus coagulans and Bacillus clausii.

4. The herbal supplement of claim 3, wherein the one or more probiotics comprises two species of Bacillus selected from the group consisting of Bacillus subtilis, Bacillus coagulans and Bacillus clausii.

5. The herbal supplement of claim 3, wherein the one or more probiotics comprises two species of Bacillus selected from the group consisting of Bacillus subtilis, Bacillus coagulans and Bacillus clausii.

6. The herbal supplement of claim 3, wherein the one or more probiotics comprises Bacillus subtilis, Bacillus coagulans and Bacillus clausii.

7. The herbal supplement of claim 6, wherein the one or more probiotics comprises Bacillus subtilis, Bacillus coagulans and Bacillus clausii at a 1:1:1 ratio.

8. The herbal supplement of claim 1, wherein the supplement is formulated in a dosage form and wherein each dosage contains the one or more probiotics in a total amount of 1×10⁸ to 1×10¹¹ cfu.

9. The herbal supplement of claim 8, wherein the supplement is formulated in a dosage form and wherein each dosage contains the one or more probiotics in a total amount of 1×10⁹ to 1×10¹⁰ cfu.

10. The herbal supplement of claim 1, wherein the herbal composition comprising condensed tannins comprises red quebracho extract.

11. The herbal supplement of claim 1, wherein the herbal composition comprises a triterpenoid saponin.

12. The herbal supplement of claim 1, wherein the triterpenoid saponin is provided in the form of an Aesculus or Satindus species plant extract.

13. The herbal supplement of claim 1, wherein the herbal composition comprises an anti-spasmodic agent.

14. The herbal supplement of claim 12, wherein the anti-spasmodic agent is provided in the form of peppermint oil.

15. The herbal supplement of claim 1, wherein the herbal supplement is formulated in a dosage form that comprises between about 20 to 500 mg of red quebracho extract; between about 100 to about 2000 mg of Aesculus hippocastanum plant extract; and between about 0.05 to about 1 ml of peppermint oil.

16. A method for treating bloating, constipation and/or weight gain in a human subject, comprising orally administering to a human subject in need thereof the herbal supplement of claim 1.

17. The method of claim 16, wherein the human subject suffers from irritable bowel syndrome, functional constipation or chronic abdominal bloating syndrome.

18. The method of claim 16, wherein the human subject suffers from obesity.

19. A method for preparing the herbal supplement of claim 1, comprising:
mixing (1) red quebracho extract, (2) an herbal composition comprising a triterpenoid saponin or an anti-spasmodic agent; and (3) one or more probiotics in endospore form to product a mixture; and
formulating the mixture into unit doses suitable for oral administration.

20. The method of claim 19, wherein the unit doses are in capsule form.
